(19) United States
(12) Patent Application Publication
(10) Pub. No.: US 2013/0281778 A1
(43) Pub. Date: Oct. 24, 2013

(54) TREATMENT INSTRUMENT FOR MEDICAL USE AND METHOD

(71) Applicant: TERUMO KABUSHIKI KAISHA, Shibuya-ku (JP)

(72) Inventors: Satoru SUEHARA, Kanagawa (JP); Riyaheh Arastoo, Kanagawa (JP); Kenichi Kumoyama, Shizuoka (JP)

(21) Appl. No.: 13/865,653

(22) Filed: Apr. 18, 2013

Related U.S. Application Data
(60) Provisional application No. 61/635,587, filed on Apr. 19, 2012.

(51) Int. Cl. A61B 1/233; A61B 1/04

(52) U.S. Cl. CPC A61B 1/233; A61B 1/04
USPC 600/104; 600/109

(57) ABSTRACT

A treatment instrument for medical use includes: an outer tube having a flexible tube at a distal portion thereof; an image information acquisition unit disposed inside the outer tube and configured to be inserted into and drawn out of the flexible tube; and a fluid conveying tube which is disposed inside the outer tube juxtaposedly with the image information acquisition unit and is insertable into and drawn out of the flexible tube in a selective manner in relation to the image information acquisition unit. The outer tube is greater than the flexible tube in diameter and is decreased in diameter, at a connection part for connection with the flexible tube, down to the diameter of the flexible tube.

This application is based on and claims priority to U.S. Provisional Application No. 61/635,587 filed on Apr. 19, 2012, the entire content of which is incorporated herein by reference.

### TECHNOLOGICAL FIELD

The present invention generally relates to a treatment instrument for medical use capable of dilating a natural ostium stenosed due to sinusitis, a method of observing the inside of a paranasal sinus, a method of dilating a stenosed part of a paranasal sinus natural ostium, and a method of treating sinusitis.

### BACKGROUND DISCUSSION

Inflammation generated in a nasal cavity due to common cold, allergy or the like may spread to a paranasal sinus, which is an intraosseous cavity adjacent to the nasal cavity. The inflammation generated in the paranasal sinus in this manner is called sinusitis. The paranasal sinus communicates with the nasal cavity through a tiny hole called the natural ostium. When edema, hypertrophy or the like of mucous membrane is generated in the vicinity of the natural ostium due to sinusitis, the natural ostium is stenosed. This makes it difficult for secretion, bacteria and the like in the paranasal sinus to be discharged to the nasal cavity, and causes ventilation disorder.

As a treatment of sinusitis, conventionally, there has commonly been practiced a surgical treatment in which the stenosed part of the natural ostium is removed by use of forceps, a drill or the like. In recent years, on the other hand, attention has been directed at a minimally invasive treatment in which the narrowed natural ostium is dilated by a balloon catheter. An example is disclosed in Japanese Application No. T-2008-513125.

Japanese Application No. T-2008-513125 describes that the natural ostium is dilated by operating a balloon catheter under endoscopic observation.

### SUMMARY

When a balloon catheter is operated endoscopically, it can be difficult to relatively easily and accurately carry out the treatment, since an operator's hands are full or the visible region is limited by the thickness (radial size) of the endoscope. In consideration of these problems, the present inventors have been researching and developing balloon catheters on which a miniaturized camera is mounted.

Here, in the case of mounting a camera on a balloon catheter, it is a common practice to dispose the camera at a distal end of the catheter and to check the forward (distal) side of the balloon, for the purpose of searching for the natural ostium. When the camera is fixedly disposed at the distal end of the catheter, however, a space for adding other functions to the distal portion of the catheter has to be secured separately. This makes the distal portion greater in diameter, thereby making it difficult to insert the balloon catheter into a living body.

The treatment instrument for medical use disclosed here facilitates relatively easy and accurate treatment of sinusitis.

The method for checking the state inside a paranasal sinus and for performing therapy of sinusitis disclosed here can be performed without any surgical procedure.

A treatment instrument for medical use includes an outer tube possessing an outer diameter, a flexible tube possessing a proximal end connected to a distal end of the outer tube by a connection portion, with the flexible tube possessing an outer diameter; an image information acquisition unit disposed inside the outer tube and configured to be inserted into and drawn out of the flexible tube; a fluid conveying tube disposed inside the outer tube in juxtaposed relation to the image information acquisition unit and configured to be inserted into and drawn out of the flexible tube in a selective manner relative to the image information acquisition unit; and the outer diameter of the outer tube at the distal end of the outer tube being greater than the outer diameter of the entire flexible tube, and the connection part being configured to possess a decreasing outer diameter that decreases from the distal end of the outer tube to the proximal end of the flexible tube.

The image information acquisition unit and the fluid conveying tube are accommodated in the outer tube of the treatment instrument for medical use. Therefore, the image information acquisition unit and the fluid conveying tube can be used while holding the treatment instrument for medical use in one hand. Consequently, easiness and accuracy of the treatment can be enhanced.

In addition, since the flexible tube is disposed at the distal portion of the outer tube, the treatment instrument for medical use can be inserted into a living body while curving the flexible tube. In addition, since the connection part for connection between the outer tube and the flexible tube is decreased in diameter down to the diameter of the flexible tube, the distal portion of the outer tube can be made small in diameter. Therefore, the flexible tube can be relatively easily inserted into a passage of a stenosed part generated in a natural ostium of a paranasal sinus. Since the image information acquisition unit and the fluid conveying tube can be selectively inserted into and drawn out of the flexible tube inserted in the stenosed part, the treatment instrument for medical use can be easily inserted into the paranasal sinus.

The treatment instrument for medical use preferably includes, inside the outer tube: a first guide tube in which the image information acquisition unit is held in an advanceable and retractable manner and by which the image information acquisition unit is guided into the flexible tube; and a second guide tube in which the fluid conveying tube is held in an advanceable and retractable manner and by which the fluid conveying tube is guided into the flexible tube.

The first guide tube for guiding the image information acquisition unit into the flexible tube and the second guide tube for guiding the fluid conveying tube into the flexible tube are disposed inside the outer tube. This helps ensure that the image information acquisition unit and the fluid conveying tube can be prevented from interfering with each other within the outer tube or being caught on the outer tube. Therefore, the image information acquisition unit and the fluid conveying tube can be rather assuredly guided into the flexible tube.

The treatment instrument for medical use disclosed here is preferably configured so that the first guide tube and the second guide tube extend up to the connection part between the outer tube and the flexible tube.

By virtue of the first and second guide tubes extending inside the outer tube up to the connection part between the outer tube and the flexible tube, the image information acquisition unit and the fluid conveying tube can be guided into the vicinity of the flexible tube by the first and second guide tubes. The connection part between the outer tube and the flexible tube is decreased in diameter down to the diameter of the flexible tube. This helps ensure that the image information acquisition unit and the fluid conveying tube can be smoothly guided along an inner surface of the connection part into the flexible tube. Therefore, image information acquisition unit and the fluid conveying tube can be more assuredly guided into the flexible tube.

The treatment instrument for medical use preferably includes position fixing means for fixing advanced/retracted positions of the image information acquisition unit and the fluid conveying tube.

The advanced/retracted positions of the image information acquisition unit and the fluid conveying tube can be fixed by the position fixing means. This makes it unnecessary for an operator to continue holding the image information acquisition unit or the fluid conveying tube. Consequently, the burden on the operator can be alleviated, and the operator is permitted to concentrate on diagnosis or therapy of a patient.

The treatment instrument for medical use preferably also includes an inflation body disposed around the outer circumference of the flexible tube and inflatable in a radial direction of the flexible tube.

The inflation body can be inflated in the stenosed part generated in the natural ostium of the paranasal sinus. Therefore, the stenosed part can be dilated and cured.

The image information acquisition unit preferably includes a biasing member configured to bias the image information acquisition unit in a curving direction.

The image information acquisition unit can thus be biased in the curving direction by the biasing member. This helps ensure that at the time of checking the inserted position of the flexible tube in the living body, there is no need for an operation of curving the image information acquisition unit, passed through the flexible tube, toward the flexible tube side. Consequently, the burden on the operator can be more alleviated.

According to another aspect, a treatment instrument for medical use includes an outer tube having a flexible tube at a distal portion of the outer tube, an image information acquisition unit disposed inside the outer tube and movable relative to the outer tube and the flexible tube to insert the image information acquisition unit into the flexible tube and to draw out the image information acquisition unit from the flexible tube, and an inflation body disposed around an outer circumferential surface of the flexible tube and inflatable in a radial outward direction of the flexible tube, and wherein the image information acquisition unit includes a biasing member configured to bias the image information acquisition unit in a curving direction.

The image information acquisition unit can be biased in the curving direction by the biasing member. This helps ensure that at the time of checking an inflated state of the inflation body in a living body, there is no need for an operation of curving the image information acquisition unit, passed through the flexible tube, toward the inflation body side. Therefore, the burden on the operator can be reduced.

The treatment instrument for medical use preferably includes, inside the outer tube, a guide tube configured to restrict variations in shape of the image information acquisition unit therein and to guide the image information acquisition unit into the flexible tube. This makes it possible to prevent the image information acquisition unit from being deformed and caught on the outer tube. Consequently, the image information acquisition unit can be assuredly guided into the flexible tube.

Another aspect involves a method of observing the inside of a paranasal sinus. The method includes inserting a flexible tube into the paranasal sinus through a nasal cavity, inserting an image information acquisition unit in the inserted flexible tube and moving the image information acquisition unit in a distal direction relative to the flexible tube to position the image information acquisition unit in the paranasal sinus, and checking a state inside the paranasal sinus based on image information about the inside of the paranasal sinus acquired by the image information acquisition unit positioned inside the paranasal sinus.

It is possible, in observing the inside of the paranasal sinus, to deform the flexible tube following up to the shape of the natural ostium of the paranasal sinus. This enables a working member such as the image information acquisition unit and the fluid conveying tube to be easily inserted into the paranasal sinus. Furthermore, the need for preparing in advance a plurality of treatment instruments or a rigid endoscope or the like as in the related art is eliminated. Therefore, the number of treatment instruments required for therapy of sinusitis can be reduced, and the effort and time required for exchange of the treatment instrument can be reduced.

In addition, since the image information acquisition unit is inserted and passed in the inserted flexible tube, the state inside the paranasal sinus can be checked without any surgical procedure.

A method of dilating a stenosed part of a paranasal sinus natural ostium according to the present invention involves inserting a flexible tube through a nasal cavity and into a paranasal sinus in which is located a stenosed part, the flexible tube including an inflatable body, inserting an image information acquisition unit into the flexible tube positioned in the paranasal sinus, moving the image information acquisition unit in a distal direction relative to the flexible tube so that a distal portion of the image information acquisition unit extends distally beyond a distal end of the flexible tube and so that the distal portion of the image information acquisition unit bends, acquiring image information about the stenosed part in the paranasal sinus by operation of the image information acquisition unit, checking a state of the stenosed part in the paranasal sinus based on the image information acquired by the image information acquisition unit, and inflating the inflation body based on a result of the checking.

The inflation body can be inflated after checking the position of the inflation body in the stenosed part on the basis of the image information supplied from the image information acquisition unit inserted in the paranasal sinus, and so the inflation body can be inflated at an appropriate position inside the stenosed part. Consequently, dilation and curing of the stenosed part can be carried out effectively.

The method preferably also includes acquiring image information on the stenosed part after inflating the inflation body, and determining whether or not the stenosed part has been dilated on the basis of the acquired image information.

Whether or not the stenosed part has been dilated is determined on the basis of the acquired image information. This makes it possible to visually inspect that the stenosed part has been dilated. Therefore, it can be assuredly confirmed that the stenosed part has been dilated, and the therapeutic result can be shown to a patient through an image.

A method of treating sinusitis comprises inserting a flexible tube into a paranasal sinus natural ostium through a nasal cavity, moving an image information acquisition unit inside the inserted flexible tube to advance the image information acquisition unit in a distal direction within the flexible tube and position the image information acquisition unit in the paranasal sinus, checking a state inside the paranasal sinus based on image information on the inside of the paranasal sinus acquired by the inserted image information acquisition unit, drawing the image information acquisition unit out of the flexible tube, moving a fluid conveying tube in the flexible tube to advance the fluid conveying tube in a distal direction within the flexible tube, and withdrawing a fluid present inside the paranasal sinus to outside by way of the fluid conveying tube positioned in the flexible tube or introducing a fluid from the outside into the paranasal sinus by way of the fluid conveying tube positioned in the flexible tube.

In conveying a fluid into or out of the paranasal sinus, the state inside the paranasal sinus can be checked on the basis of the acquired image information on the inside of the paranasal sinus. Therefore, it is possible to carry out an effective therapy according to the state inside the paranasal sinus.

### BRIEF DESCRIPTION OF THE DRAWINGS

FIG. 1 is a lateral view showing, partly in cross-section, a treatment instrument for medical use according to one embodiment disclosed by way of example here.

FIGS. 2A and 2B are views, partly in cross-section, illustrating a grip section of the treatment instrument for medical use.

FIG. 3 is a cross-sectional view of an image information acquisition unit accommodated in the treatment instrument for medical use.

FIGS. 4A and 4B are views which illustrate the shape of a distal portion of a biasing member disposed in the image information acquisition unit.

FIG. 5A is an illustration of a state in use of the treatment instrument for medical use.

FIG. 5B is an illustration of a state in use of the treatment instrument for medical use.

FIG. 5C is an illustration of a state in use of the treatment instrument for medical use.

FIG. 5D is an illustration of a state in use of the treatment instrument for medical use.

FIG. 5E is an illustration of a state in use of the treatment instrument for medical use.

FIG. 5F is an illustration of a state in use of the treatment instrument for medical use.

### DETAILED DESCRIPTION

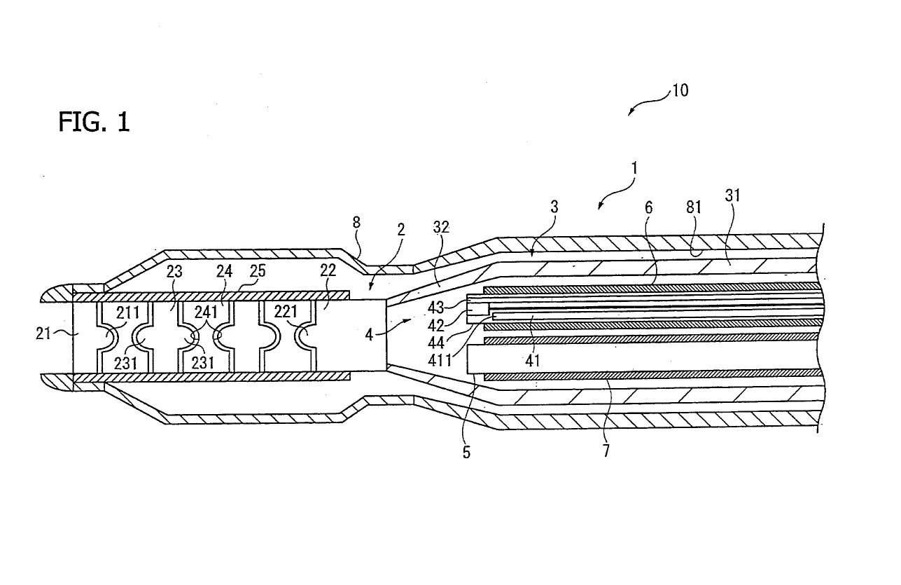

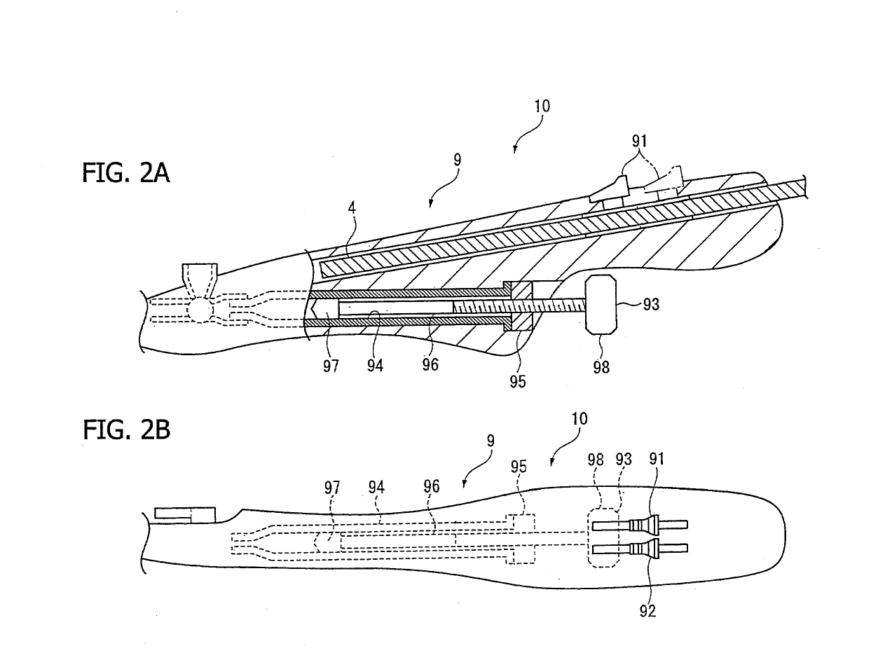

As shown in FIGS. 1, 2A and 2B, a treatment instrument 10 for medical use according to one embodiment, representing an example of the medical use treatment instrument disclosed here, includes an introduction section 1 (FIG. 1) to be introduced into a living body, and a grip section 9 (FIGS. 2A and 2B) disposed on the proximal side of the introduction section 1.

In FIG. 1, the introduction section 1 includes: an outer tube 3, a flexible tube 2 at a distal portion of the outer tube 3; an endoscope 4 as an image information acquisition unit disposed inside the outer tube 3 and configured to be inserted into and drawn out of the flexible tube 2; a suction tube 5 as a fluid conveying tube disposed inside the outer tube 3 juxtaposedly (in side-by-side relation) with the endoscope 4 and configured to be inserted into and drawn out of the flexible tube 2 in a selective manner in relation to the endoscope 4; a first guide tube 6 as a guide tube configured to guide the endoscope 4 into the flexible tube 2; a second guide tube 7 configured to guide the suction tube 5 into the flexible tube 2; and an inflation body 8 disposed around an outer circumference of the flexible tube 2 and configured to inflate in the radial direction of the flexible tube 2.

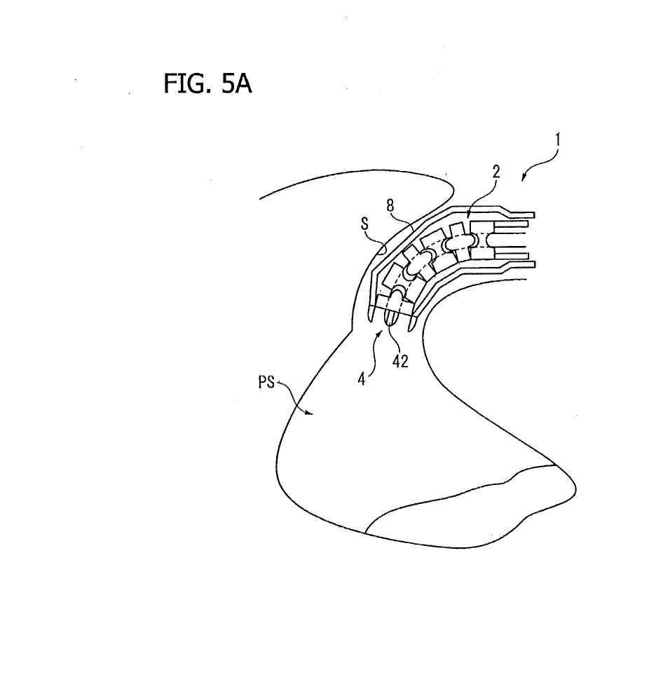

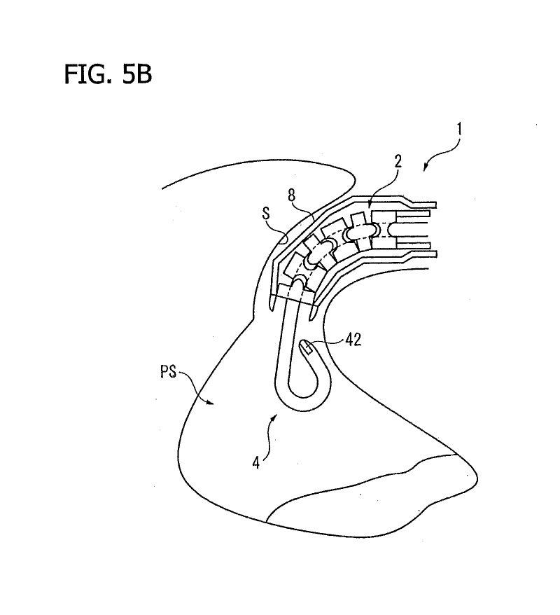

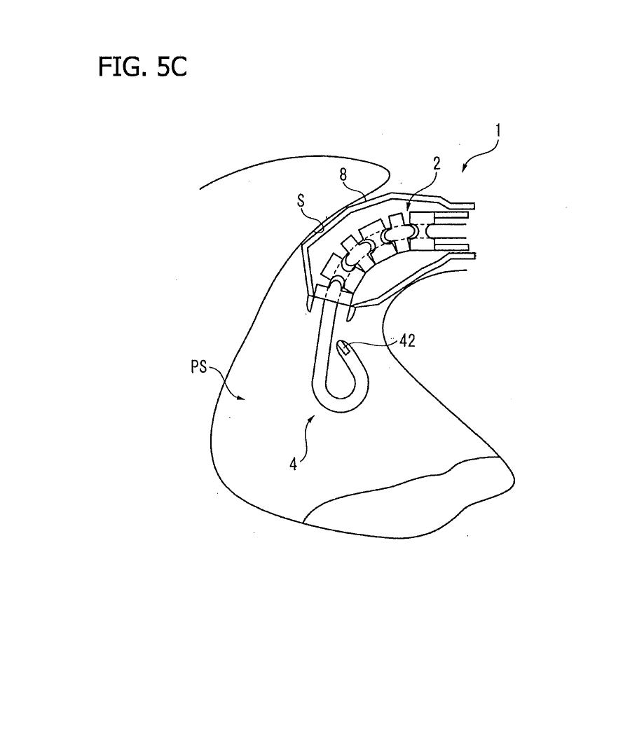

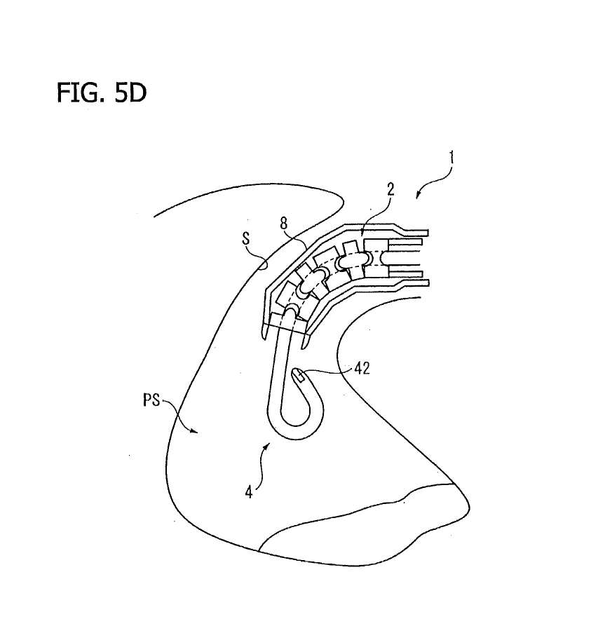

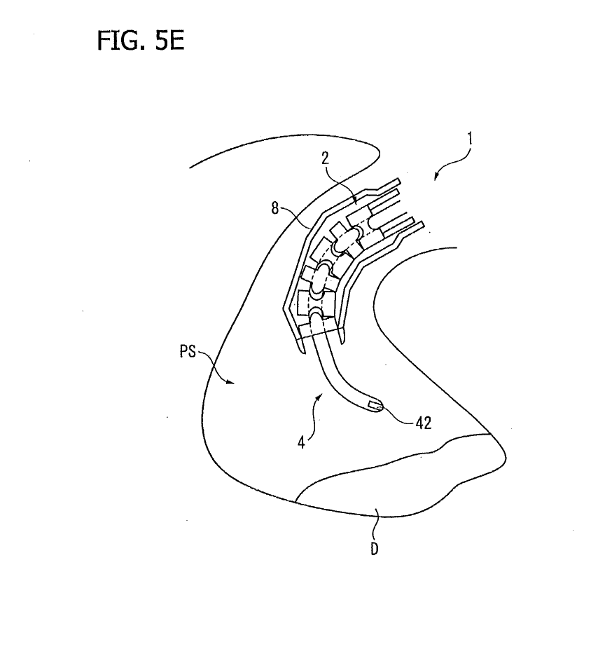

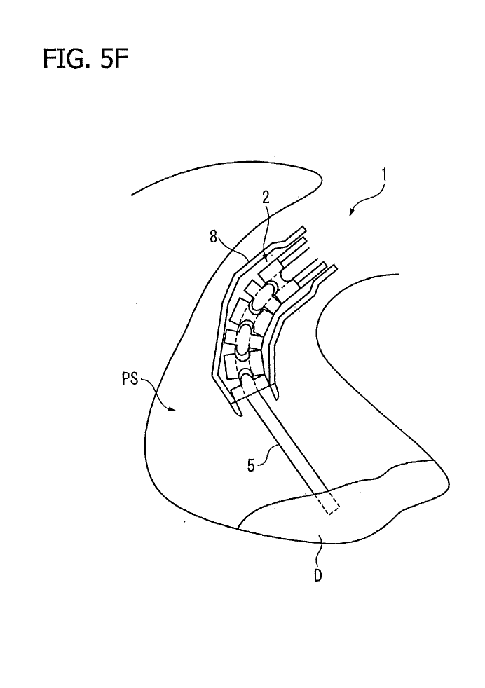

The flexible tube 2 includes a plurality of tubular elements 21, 22, 23, 24, which are turnable relative to one another and are connected to one another in an axial direction. That is, the tubular elements are turnable relative to one another so that the distal end of the flexible tube can be bent such as in the manner shown in FIG. 5A. Operating means such as a wire is connected to the tubular element 21 on the distal side (distal end) of the flexible tube 2. The flexible tube 2 can be curved, and the curved position of the flexible tube 2 can be fixed, by the operating means. In addition, the outer circumference of the flexible tube 2 is covered, in close contact, by a flexible tubular member 25 such as an elastic member, so that a fluid being fed to the inflation body 8 will not flow into the inside of the flexible tube 2 through gaps between the tubular elements 21 to 24.

The tubular element 21 on the distal side and the tubular element 22 on the proximal side have turning shaft sections 211 and 221, respectively, projecting in a connecting direction (the axial direction of each of the tubular elements 21 and 22) from an end edge on one side in the connecting direction. Between the tubular element 21 on the distal side and the tubular element 22 on the proximal side, the tubular elements 23 and 24 having different shapes are alternately arranged.

The tubular element 23 has turning shaft sections 231 projecting in the connecting direction (the axial direction of the tubular element 23) from end edges on both sides (proximal and distal sides/ends) in the connecting direction.

On the other hand, the tubular element 24 has turning support sections 241 where end edges on both sides (proximal and distal sides/ends) in the connecting direction (the axial direction of the tubular element 24) are recessed. The turning shaft sections 211, 221 and 231 of the tubular elements 21 to 23 are supported by the turning support sections 241.

The turning shaft sections 211, 221 and 231 and the turning support sections 241 are disposed in pairs at positions symmetric about center axes of the tubular elements 21 to 24, respectively.

The outer tube 3 includes a tubular accommodation section 31 in which the guide tubes 6 and 7 are accommodated, and a conical decreasing-diameter section 32 which constitutes a connection part between the accommodation section 31 (distal end of the outer tube 31) and the flexible tube 2 (proximal end of the flexible tube 2) and which decreases in diameter (both inner and outer diameters in the illustrated embodiment) from the diameter of the accommodation section 31 to the diameter of the flexible tube 2.

The flexible tube 2 and the outer tube 3 as above-mentioned are obtained, for example, by a method in which a portion on the distal side relative to the decreasing-diameter section 32, of a stainless steel-made hollow cylindrical member having the decreasing-diameter section 32, is subjected to laser beam processing. Where laser beam processing is used, the introduction section 1 in the state in which the tubular elements 21 to 24 of the flexible tube 2 are connected with one another can be relatively easily obtained by simply cutting the hollow cylindrical member by use of a laser beam.

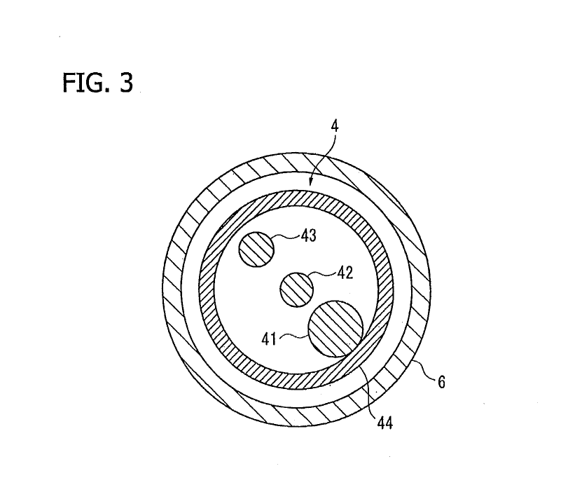

As shown in FIG. 3 also, the endoscope 4 includes: a biasing member 41 for biasing the endoscope 4 in a curving direction; an image sensing unit 42 having an image sensing device such as a CCD (Charge Coupled Device) image sensor; a light transmission section 43 such as an optical fiber for transmitting light toward a distal portion of the endoscope 4; and an outer cylinder 44 for accommodating these components 41 to 43. The endoscope 4 is so configured that it can be cured by operating means such as a wire connected to a distal portion of the endoscope. The image sensing unit 42 is not restricted to the image sensing unit described above. For example, it may be a digital video camera using other image sensing device such as a CMOS (Complementary Metal-Oxide Semiconductor) image sensor, an image fiber for acquisition and transmission of an image by utilizing optical fiber, or an image sensing system for transmission of an image by an optical system including an objective lens and a plurality of relay lenses.

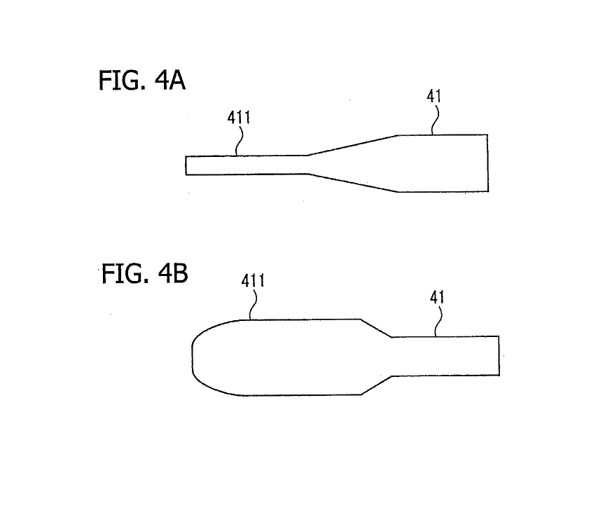

The biasing member 41 is formed from a superelastic alloy such as nickel-titanium alloys, or from a shape memory alloys, and has a previously reshaped curved portion which turns back toward the proximal side in a condition where no external force is exerted on the biasing member. In other words, when no force is applied to the biasing member, the biasing member will bend or curve. As shown in FIG. 4A and FIG. 4B, a distal portion 411 of the biasing member 41 is formed in the shape of a thin sheet which is thinned relative to the proximal side in lateral view and which is broadened relative to the proximal side in plan view. With the distal portion 411 formed in such a shape, the distal portion 411 can be inhibited or prevented from damaging the outer cylinder 44.

The suction tube 5 is a pipeline for sucking solid matter, fluid or the like present in a living body, and is connected to a suction device such as a suction pump.

The first and second guide tubes 6 and 7 are accommodated in the accommodation section 31 of the outer tube 3, and extend up to the decreasing-diameter section 32 of the outer tube 3. That is, the first and second guide tubes 6 and 7 axially overlap a portion of the outer tube located proximal of the decreasing-diameter section 32 (connection part) of the outer tube 3, and extend up to a location at which the distal end of the outer tube 3 is connected to the decreasing-diameter section 32 (connection part) of the outer tube 3.

The endoscope 4 is held in the first guide tube 6 in an advanceable and retractable manner, and in the first guide tube 6 guides the endoscope 4 into the flexible tube 2 while restricting the variation in shape of the endoscope 4 by the biasing member 41.

The suction tube 5 is held in the second guide tube 7 in an advanceable and retractable manner.

The inflation body 8 is formed from a flexible material such as a polymer. The inside of the inflation body 8 communicates with a channel 81 disposed around the outer tube 3, and is so configured that the inflation body 8 is radially inflated when a fluid is introduced into the inflation body 8 through the channel 81.

As shown in FIG. 2A and FIG. 2B, the grip section 9 includes: a first operating section 91 (representing an example of position fixing means for fixing the position of the endoscope 4) by which the endoscope 4 is inserted into and drawn out of the flexible tube 2 and by which the advanced/retracted position of the endoscope 4 is fixed; a second operating section 92 (representing an example of position fixing means for fixing the position of the suction tube 5) by which the suction tube 5 is inserted into and drawn out of the flexible tube 2 and by which the advanced/retracted position of the suction tube 5 is fixed; and a third operating section 93 by which the inflation body 8 is inflated or contracted and by which the inflated state of the inflation body 8 is maintained.

The first operating section 91 and the second operating section 92 hold the endoscope 4 and the suction tube 5 respectively inside the grip section 9. These operating sections 91 and 92 are slidable toward the outer tube 3, and can each be fixed at a position indicated by solid line in FIG. 2A and a position indicated by alternate long and short dash line in FIG. 2A. Specifically, when each of the operating sections 91 and 92 is located in the position of solid line, the endoscope 4 or the suction tube 5 is inserted and located in the flexible tube 2, whereas when each of the operating sections 91 and 92 is located in the position of alternate long and short dash line, the endoscope 4 or the suction tube 5 is drawn out or located outside of the flexible tube 2.

The third operating section 93 is so disposed that it can be advanced into and retracted from a fluid introduction passage 94 communicating with the channel 81 for the inflation body 8. With the third operating section 93 advanced or retracted, the inflation body 8 is inflated or contracted. Specifically, the third operating section 93 includes: a rod 96 screw engaged with a threaded hole formed in a plug member 95 for plugging up the fluid introduction passage 94; a seal member 97 disposed in the fluid introduction passage 94 on one end of the rod 96 and configured to seal the fluid introduction passage 94; and a knob section 98 disposed outside of the fluid introduction passage 94 on the other end of the rod 96.

Examples of a manner of use or operation of the treatment instrument 10 for medical use as above-described, procedures for diagnosis of a paranasal sinus and therapy of sinusitis by use of the treatment instrument 10 for medical use will be described below.

First, an operator inserts the introduction section 1 of the treatment instrument 10 for medical use through a naris, and guides the flexible tube 2 and the inflation body 8 to a stenosed part S of a natural ostium of a paranasal sinus PS, while curving the flexible tube 2, as shown in FIG. 5A (Procedure 1).

Next, the operator operates the first operating section 91 so that the endoscope 4 protrudes from (extends distally beyond) the flexible tube 2, and to insert the image sensing unit 42 of the endoscope 4 into the paranasal sinus PS (Procedure 2). As a result, as shown in FIG. 5B, the endoscope 4 is curved by the biasing force of the biasing member 41, and the image sensing unit 42 is directed toward the stenosed part S side. In this condition, the operator checks the state of the stenosed part S and the position of the inflation body 8 inside the stenosed part S, based on image information acquired by the image sensing unit 42 (Procedure 3). Thus, the image sensing unit 42 of the endoscope 4 can be directed to the stenosed part S, without an operation for curving the endoscope 4 toward the stenosed part S side by use of a complicated mechanism. Therefore, the operator can easily observe the state of the stenosed part S generated at the natural ostium of the paranasal sinus PS, and can easily observe whether or not the inflation body 8 is disposed in a position including the stenosed part S.

When it is confirmed that the inflation body 8 is located in the stenosed part S, the operator operates the third operating section 93 so as to introduce a fluid into the inflation body 8, thereby inflating the inflation body 8, as shown in FIG. 5C (Procedure 4). In this instance, the image sensing unit 42 of the endoscope 4 is kept facing toward the stenosed part S side by the biasing force of the biasing member 41; therefore, the manner in which the inflation body 8 is inflated can be observed, based on the image information acquired by the image sensing unit 42.

Then, when the inflation body 8 is contracted as shown in FIG. 5D, the operator acquires image information on the stenosed part S after the inflation of the inflation body 8 (Procedure 5), and determines whether or not the stenosed part S has been dilated on the basis of the image information (Procedure 6). In this instance, it can be confirmed by visual inspection that the stenosed part S has been dilated.

Thereafter, as shown in FIG. 5E, the operator operates the first operating section 91 so as to direct the endoscope 4 in a direction opposite to the biasing direction of the biasing member 41 (in such a direction that the endoscope 4 becomes rectilinear), thereby directing the image sensing unit 42 toward a bottom portion of the paranasal sinus PS. Then, the operator checks the state inside the paranasal sinus PS on the basis of image information on the inside of the paranasal sinus PS acquired by the image sensing unit 42 (Procedure 7). In addition, by repeating the operations of advancing and retracting the first operating section 91, it is possible to selectively change the angle of a distal end of the endoscope 4, so that the inside of the paranasal sinus PS can be observed widely.

In this instance, when undrained collection D such as mucus or solid matter formed by solidification of mucus is present inside the paranasal sinus PS, the operator operates the first operating section 91 so as to draw the endoscope 4 out of the flexible tube 2 and back into the outer tube 3 (Procedure 8). Thereafter, the operator operates the second operating section 92 so as to insert and pass the suction tube 5 in the flexible tube 2, as shown in FIG. 5F (Procedure 9), and sucks the undrained collection D by the suction tube 5 (Procedure 10). In addition, by introducing a fluid such as physiological saline into the suction tube 5, it is also possible to wash the inside of the paranasal sinus PS with the fluid. In addition, by introducing a fluid such as physiological saline into the suction tube 5 by applying pressure (pressurization), it is also possible to remove the mucosa or tissues from the inside of the paranasal sinus PS with the pressurized fluid.

In a case where only observation of the inside of the paranasal sinus PS is to be conducted, it is possible to only carry out the above-mentioned Procedures 1, 2 and 7.

In a case where only dilation of the stenosed part S generated at the natural ostium of the paranasal sinus PS is to be performed, it is possible to carry out the above-mentioned Procedures 1 to 4. Further, with the Procedures 5 and 6 carried out subsequently, it is possible to visually check whether the stenosed part S has been dilated; thus, it can be assuredly confirmed that the stenosed part S has been dilated.

Furthermore, in a case where only therapy of sinusitis is to be conducted, it suffices to carry out the above-mentioned Procedures 1, 2, and 7 to 10.

The medical use treatment instrument disclosed here provides the following effects.

Since the flexible tube 2 is disposed at the distal portion of the outer tube 3 in which the endoscope 4 and the suction tube 5 are accommodated, the introduction section 1 of the treatment instrument 10 for medical use can be inserted into a living body while curving the flexible tube 2. In addition, since the connection part between the outer tube 3 and the flexible tube 2 is decreased in diameter down to the diameter of the flexible tube 2, the distal portion of the introduction section 1 can be made small in diameter. Consequently, it is possible to facilitate insertion of the treatment instrument 10 for medical use into a living body, while adopting a configuration in which the endoscope 4 and the suction tube 5 can be selectively inserted into and drawn out of the flexible tube 2.

In observing the inside of the paranasal sinus PS, the flexible tube 2 can be deformed to follow the shape of the natural ostium of the paranasal sinus PS for the purpose of inserting the flexible tube 2 into the paranasal sinus PS, so that the introduction section 1 of the treatment instrument 10 for medical use can be easily inserted into the paranasal sinus PS. Further, since the need to prepare a plurality of treatment instruments, a hard endoscope or the like as used previously is eliminated, the number of treatment instruments necessary for curing sinusitis can be reduced, and the effort and time required for exchange of the treatment instrument can be reduced.

Since the endoscope 4 is inserted and passed in the inserted flexible tube 2, the state inside the paranasal sinus PS can be checked without any surgical procedure.

Since the inflation body 8 is inflated after confirming the position of the inflation body 8 in the stenosed part S at the natural ostium of the paranasal sinus PS, based on the image information sent from the endoscope 4 inserted in the paranasal sinus PS, the inflation body 8 can be inflated at an appropriate position in the stenosed part S. Therefore, the stenosed part S can be dilated and cured effectively.

In conveying a fluid into or out of the paranasal sinus PS, the state inside the paranasal sinus PS can be checked, based on the acquired image information on the inside of the paranasal sinus PS. This ensures that an effective therapy can be carried out according to the state inside the paranasal sinus PS.

The medical use treatment instrument disclosed here is not limited to the above-described embodiment, and modifications, improvements and the like which permit attainment of functions and operations similar to those associated with the medical use treatment instrument disclosed here are embraced.

For instance, while the inflation body 8 has been provided in the above-described embodiment, the inflation body 8 is indispensable only in the case of dilating and curing a stenosed part S; thus, the treatment instrument 10 for medical use may be configured without providing the inflation body 8.

While the endoscope 4 has included the biasing member 41 in the above-described embodiment, the biasing member 41 may be omitted insofar as the endoscope 4 is so configured that it can be curved by operating means such as a wire. In addition, the biasing member 41 may be formed from a material other than nickel-titanium alloy or shape memory alloy, so long as it can bias the endoscope 4 in a curving direction.

While the flexible tube 2 has been formed of a metal such as stainless steel in the above-described embodiment, the flexible tube 2 may be formed from other material such as resins or other metals than the above-mentioned, insofar as the material is flexible.

In place of the suction tube 5, other members such as a guide wire or a forceps may be held inside the second guide tube 7 in an advanceable and retractable manner.

While the treatment instrument 10 for medical use has been used for observation of the inside of a paranasal sinus, dilation of a stenosed part generated at a natural ostium of the paranasal sinus, or curing of sinusitis, the treatment instrument 10 for medical use may be used for observation and/or therapy of other parts in a living body.

The present invention is applicable not only to a treatment instrument for medical use which are used for diagnosis and therapy of a paranasal sinus but also to a treatment instrument for medical use which are used for other diagnosis or therapy not involving any surgical procedure.

The detailed description above describes a treatment instrument for medical use according to an embodiment disclosed by way of example. The invention here is not limited, however, to the precise embodiment and variations described above and illustrated in the drawing figures. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A treatment instrument for medical use comprising:
an outer tube possessing an outer diameter;
a flexible tube possessing a proximal end connected to a distal end of the outer tube by a connection portion, the flexible tube possessing an outer diameter;
an image information acquisition unit disposed inside the outer tube and configured to be inserted into and drawn out of the flexible tube;
a fluid conveying tube disposed inside the outer tube in juxtaposed relation to the image information acquisition unit and configured to be inserted into and drawn out of the flexible tube in a selective manner relative to the image information acquisition unit; and
the outer diameter of the outer tube at the distal end of the outer tube being greater than the outer diameter of the entire flexible tube, and the connection part being configured to possess a decreasing outer diameter that decreases from the distal end of the outer tube to the proximal end of the flexible tube.

2. The treatment instrument for medical use according to claim 1, further comprising, inside the outer tube:
a first guide tube in which the image information acquisition unit is positioned in a manner permitting advancing and retracting movement of the image information acquisition unit and by which the image information acquisition unit is guided into the flexible tube; and
a second guide tube in which the fluid conveying tube is positioned in a manner permitting advancing and retracting movement of the fluid conveying tube and by which the fluid conveying tube is guided into the flexible tube.

3. The treatment instrument for medical use according to claim 2, wherein the first guide tube and the second guide tube axially overlap a portion of the outer tube located proximal to the connection part and extend up to connection part between the outer tube and the flexible tube.

4. The treatment instrument for medical use according to claim 1, further comprising position fixing means for fixing advanced/retracted positions of the image information acquisition unit and the fluid conveying tube.

5. The treatment instrument for medical use according to claim 1, further comprising an inflation body disposed around an outer circumference of the flexible tube and inflatable in a radial direction of the flexible tube.

6. The treatment instrument for medical use according to claim 1, wherein the image information acquisition unit includes a biasing member configured to bias the image information acquisition unit in a curving direction.

7. A treatment instrument for medical use comprising:
an outer tube having a flexible tube at a distal portion of the outer tube;
an image information acquisition unit disposed inside the outer tube and movable relative to the outer tube and the flexible tube to insert the image information acquisition unit into the flexible tube and to draw out the image information acquisition unit from the flexible tube;
an inflation body disposed around an outer circumferential surface of the flexible tube and inflatable in a radial outward direction of the flexible tube; and
the image information acquisition unit including a biasing member configured to bias the image information acquisition unit in a curving direction.

8. The treatment instrument for medical use according to claim 7, further comprising a guide tube positioned in the outer tube, the image information acquisition unit being positioned in the guide tube so that the guide tube restricts variations in shape of the image information acquisition unit and guides the image information acquisition unit into the flexible tube.

9. The treatment instrument for medical use according to claim 8, further comprising a fluid conveying tube disposed inside the outer tube in side-by-side relation to the image information acquisition unit and movable relative to the flexible tube and the outer tube so that the fluid conveying tube is insertable into and movable out of the flexible tube in a selective manner relative to the image information acquisition unit.

10. The treatment instrument for medical use according to claim 9, wherein the guide tube is one guide tube, and further comprising another guide tube located inside the outer tube and in which is movably positioned the fluid conveying tube.

11. The treatment instrument for medical use according to claim 7, further comprising a fluid conveying tube inside the outer tube and positioned in side-by-side relation to the image information acquisition unit, the fluid conveying tube being movable relative to the flexible tube and the outer tube so that the fluid conveying tube is insertable into and movable out of the flexible tube in a selective manner relative to the image information acquisition unit.

12. A method of observing the inside of a paranasal sinus comprising:
inserting a flexible tube into the paranasal sinus through a nasal cavity;
inserting an image information acquisition unit in the inserted flexible tube and moving the image information acquisition unit in a distal direction relative to the flexible tube to position the image information acquisition unit in the paranasal sinus; and
checking a state inside the paranasal sinus based on image information about the inside of the paranasal sinus acquired by the image information acquisition unit positioned inside the paranasal sinus.

13. The method of observing the inside of a paranasal sinus according to claim 12, further comprising a biasing member that causes the image information acquisition unit to curve, and wherein the moving of the image information acquisition unit in the distal direction relative to the flexible tube includes moving the image information acquisition unit in the distal direction relative to the flexible tube until a distal portion of the image information acquisition unit is positioned distally beyond a distal end of the flexible tube, the biasing member causing the distal portion of the image information acquisition unit positioned distally beyond the distal end of the flexible tube to curve.

14. The method of observing the inside of a paranasal sinus according to claim 12, further comprising checking a position of the image information acquisition unit inside the paranasal sinus based on the image information about the inside of the paranasal sinus acquired by the image information acquisition unit positioned inside the paranasal sinus.

15. A method of dilating a stenosed part of a paranasal sinus natural ostium comprising:
inserting a flexible tube through a nasal cavity and into a paranasal sinus in which is located a stenosed part, the flexible tube including an inflatable body;
inserting an image information acquisition unit into the flexible tube positioned in the paranasal sinus;
moving the image information acquisition unit in a distal direction relative to the flexible tube so that a distal portion of the image information acquisition unit extends distally beyond a distal end of the flexible tube and so that the distal portion of the image information acquisition unit bends;
acquiring image information about the stenosed part in the paranasal sinus by operation of the image information acquisition unit;
checking a state of the stenosed part in the paranasal sinus based on the image information acquired by the image information acquisition unit; and
inflating the inflation body based on a result of the checking.

16. The method of dilating the stenosed part of the paranasal sinus natural ostium according to claim 15, further comprising checking a position of the inflation body in the paranasal sinus based on the image information acquired by the image information acquisition unit.

17. The method of dilating the stenosed part of the paranasal sinus natural ostium according to claim 15, wherein the distal portion of the image information acquisition unit bends automatically by virtue of a biasing force applied to the distal portion of the image information acquisition unit.

18. The method of dilating the stenosed part of the paranasal sinus natural ostium according to claim 15, further comprising acquiring additional image information about the stenosed part in the paranasal sinus after inflating the inflation body, and further comprising determining whether or not the stenosed part has been dilated based on the additional acquired image information.

19. A method of treating sinusitis comprising:
inserting a flexible tube into a paranasal sinus natural ostium through a nasal cavity;
moving an image information acquisition unit inside the inserted flexible tube to advance the image information acquisition unit in a distal direction within the flexible tube and position the image information acquisition unit in the paranasal sinus;
checking a state inside the paranasal sinus based on image information on the inside of the paranasal sinus acquired by the inserted image information acquisition unit;
drawing the image information acquisition unit out of the flexible tube;
moving a fluid conveying tube in the flexible tube to advance the fluid conveying tube in a distal direction within the flexible tube; and
withdrawing a fluid present inside the paranasal sinus to outside by way of the fluid conveying tube positioned in the flexible tube or introducing a fluid from the outside into the paranasal sinus by way of the fluid conveying tube positioned in the flexible tube.

20. A method of treating sinusitis according to claim 19, wherein the moving of the image information acquisition unit inside the inserted flexible tube to advance the image information acquisition unit in the distal direction within the flexible tube and position the image information acquisition unit in the paranasal sinus also comprises bending a distal portion of the image information acquisition unit when the distal portion of the image information acquisition unit is located in the paranasal sinus.
